(19) United States
(12) Patent Application Publication
(10) Pub. No.: US 2023/0372642 A1
(43) Pub. Date: Nov. 23, 2023

(54) DEVICES, ASSEMBLIES, AND METHODS FOR DELIVERING AGENTS

(71) Applicant: Boston Scientific Scimed Inc., Maple Grove, MN (US)

(72) Inventors: John B. GOLDEN, Norton, MA (US); Ryan EVERS, Billerica, MA (US)

(73) Assignee: Boston Scientific Scimed Inc., Maple Grove, MN (US)

(21) Appl. No.: 18/319,720

(22) Filed: May 18, 2023

Related U.S. Application Data
(60) Provisional application No. 63/343,754, filed on May 19, 2022.

(51) Int. Cl. A61M 11/06; A61M 39/22; A61M 11/00

(52) U.S. Cl. CPC A61M 11/06; A61M 39/22; A61M 11/001; A61M 2202/064

(57) ABSTRACT

A valve assembly for a medical device that includes a body having an inlet that is in fluid communication with a source of fluid, and an outlet that is in fluid communication with a delivery conduit of the medical device. The valve assembly includes a shuttle configured to move within the body between a first and second position to control delivery of fluid from the inlet to the outlet. In the first position, the shuttle is configured to maintain the fluid at a first pressure within the body and seal the outlet from the inlet, such that the delivery conduit is isolated from the fluid at the first pressure. In the second position, the shuttle is configured to maintain the fluid at a second pressure within the body and fluidly couple the outlet to the inlet, such that the delivery conduit receives the fluid at the second pressure.

### CROSS-REFERENCE TO RELATED APPLICATIONS

This application claims the benefit of priority of U.S. Provisional Patent Application No. 63/343,754, filed May 19, 2022, the entirety of which is incorporated herein by reference.

### TECHNICAL FIELD

Various aspects of this disclosure relate generally to devices and methods for delivering agents. More specifically, in embodiments, this disclosure relates to devices for delivery of powdered agents, such as hemostatic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines. During an endoscopic procedure, a user inserts a sheath of an endoscope into a body lumen of a patient. The user utilizes a handle of the endoscope to control the endoscope during the procedure. Tools may be passed through a working channel of the endoscope via, for example, a port in the handle, to deliver treatment at the procedure site near a distal end of the endoscope. The procedure site is remote from the user.

To achieve hemostasis at the remote site, a hemostatic agent may be delivered by a device inserted into the working channel of the endoscope. Agent delivery may be achieved, for example, through mechanical systems. Such systems, however, may require numerous steps or actuations to achieve delivery, may not achieve a desired rate of agent delivery or a desired dosage of agent, may result in the agent clogging portions of the delivery device, may result in inconsistent dosing of the agent, and/or may not result in the agent reaching the treatment site deep within the gastrointestinal tract. The current disclosure may solve one or more of these issues or other issues in the art.

### SUMMARY

Each of the aspects disclosed herein may include one or more of the features described in connection with any of the other disclosed aspects.

According to an example, a valve assembly for a medical device that includes a body having an inlet that is in fluid communication with a source of fluid, and an outlet that is in fluid communication with a delivery conduit of the medical device; and a shuttle configured to move within the body between a first position and a second position to control delivery of the fluid from the inlet to the outlet; wherein, in the first position, the shuttle is configured to maintain the fluid at a first pressure within the body and seal the outlet from the inlet, such that the delivery conduit is isolated from the fluid received from the source at the first pressure; and wherein, in the second position, the shuttle is configured to maintain the fluid at a second pressure within the body and fluidly couple the outlet to the inlet, such that the delivery conduit receives the fluid from the source at the second pressure, the second pressure being less than the first pressure.

Any of the valve assemblies described herein may include any of the following features. The valve assembly including a piercing element configured to move within the shuttle between a retracted position and an extended position to fluidly couple the inlet with the source of fluid. The piercing element includes a needle or a lance having a sharp tip. The valve assembly is configured to receive the fluid from the source through the inlet when the shuttle is moved from the first position to the second position, and the piercing element is positioned in the retracted position; and wherein the valve assembly is configured to inhibit delivery of the fluid from the inlet to the outlet when the shuttle is moved from the second position to the first position, and the piercing element is positioned in the extended position. In a first configuration of the valve assembly: the shuttle is positioned at the first position; the piercing element is in the retracted position and separated from the source of fluid; and the inlet is not in fluid communication with the outlet such that the fluid is maintained in the source of the fluid at the first pressure. In a second configuration of the valve assembly: the shuttle is positioned at the second position; the piercing element is in the retracted position and in contact with the source of fluid; and the inlet is in fluid communication with the outlet such that the fluid is directed to the outlet at the second pressure. In a third configuration of the valve assembly: the shuttle is positioned at the first position; the piercing element is in the extended position and in contact with the source of fluid; and the inlet is not in fluid communication with the outlet such that the fluid is maintained in the body at the first pressure and the second pressure. The body is in fluid communication with a regulator assembly configured to convert the fluid from the first pressure to the second pressure. The shuttle includes a gasket positioned about an exterior surface of the shuttle. In a first configuration of the valve assembly: the shuttle is positioned at the first position; and the gasket is positioned between the inlet and the outlet to fluidly isolate the delivery conduit from the source of fluid. In a second configuration of the valve assembly: the shuttle is positioned at the second position; and the gasket is not positioned between the inlet and the outlet to fluidly couple the delivery conduit to the source of fluid. The body includes a channel configured to receive the shuttle, and a ledge extending radially-inward into the channel; wherein the shuttle includes a flange extending radially-outward from an exterior surface of the shuttle. The flange is configured to engage the ledge when the shuttle is in the first position relative to the channel to position the gasket between the inlet and the outlet, thereby fluidly isolating the delivery conduit from the source of fluid. The flange is configured to disengage the ledge when the shuttle is in the second position relative to the channel to position the gasket outside of the inlet and the outlet, thereby fluidly coupling the delivery conduit from the source of fluid. The valve assembly including a biasing mechanism coupled to the shuttle, the biasing mechanism being configured to move the shuttle from the second position to the first position.

According to another example, a device for delivering an agent includes an enclosure configured to store an agent; a pressurized fluid source configured to store a pressurized fluid; a valve assembly, including: a body having a first channel; a shuttle configured to move within the first channel between a first position and a second position to fluidly couple the pressurized fluid source to the enclosure, the shuttle having a second channel; and a piercing element configured to move within the second channel between a retracted position and an extended position to fluidly couple the pressurized fluid source to the body; wherein the valve assembly is configured to selectively release the pressurized fluid from the pressurized fluid source when moving the shuttle to the second position and the piercing element to the retracted position, or moving the shuttle to the first position and the piercing element to the extended position. The valve assembly includes a biasing mechanism configured to bias the shuttle toward the first position when in the second position.

Any of the valve assemblies described herein may include any of the following features. The pressurized fluid from the pressurized fluid source is configured to bias the shuttle toward the first position when in the second position. The valve assembly is configured to selectively inhibit delivery of the pressurized fluid from the pressurized fluid source to the enclosure when moving the shuttle from the second position to the first position, and the piercing element to the extended position.

According to a further example, a method for delivering a fluid from a medical device includes moving a shuttle relative to a valve body from a first position that is offset from a source of fluid coupled to the valve body to a second position that is in contact with the source of fluid, thereby releasing the fluid into the valve body; directing the fluid from the source to an inlet of the valve body based on the second position of the shuttle, wherein a pressure of the fluid is adjusted from a first pressure to a second pressure that is less than the first pressure when directed through the inlet; directing the fluid from the inlet to an outlet of the valve body based on the second position of the shuttle, wherein the outlet is in fluid communication with a delivery conduit of the medical device such that the fluid at the second pressure is received at the delivery conduit; moving the shuttle relative to the valve body from the second position to the first position, wherein the shuttle is configured to continue releasing the fluid into the valve body when in the first position; and inhibiting the fluid from entering the outlet of the valve body based on the first position of the shuttle, such that the fluid at the first pressure and the second pressure is maintained within the valve body.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows an exemplary delivery device.

FIG. 2 shows a partial perspective view of an exemplary valve assembly of the delivery device of FIG. 1.

FIG. 3 shows a perspective view of the valve assembly of FIG. 2.

FIG. 4 shows a cross-sectional side view of the valve assembly of FIG. 2, taken along line 4-4 of FIG. 3.

FIG. 5A shows a cross-sectional view of the valve assembly of FIG. 2 in a first configuration.

FIG. 5B shows a cross-sectional view of the valve assembly of FIG. 2 in a second configuration.

FIG. 5C shows a cross-sectional view of the valve assembly of FIG. 2 in a third configuration.

FIG. 6A shows a cross-sectional side view of another exemplary valve assembly a first configuration.

FIG. 6B shows a cross-sectional side view of the valve assembly of FIG. 6A in a second configuration.

FIG. 6C shows a cross-sectional side view of the valve assembly of FIG. 6A in a third configuration.

FIG. 7 shows a perspective view of another exemplary valve assembly.

FIG. 8 shows a cross-sectional view of the valve assembly of FIG. 7, taken along line 8-8 of FIG. 7.

### DETAILED DESCRIPTION

Embodiments of this disclosure relate to dispensing devices having valve assemblies for selectively releasing a pressurized fluid for delivering an agent (e.g., a powdered agent) to a site of a medical procedure. The valve assembly may include a movable piercing element for actuating a pressurized medium source (e.g., a gas canister) from which the pressurized fluid (e.g., a gas) may be released prior to encountering the agent. The agent may be received within an enclosure of the dispending device, and in fluid communication with the pressurized fluid through an outlet of the valve assembly. Accordingly, when the pressurized fluid is selectively released from the pressurized fluid source by the valve assembly, it may travel toward the outlet and enter the enclosure to agitate the agent prior to delivery to a target site of the medical procedure. Aspects of the dispensing device and valve assembly, such as the movable piercing element and outlet, may facilitate a fluidization of the agent with the flow of pressurized fluid prior to the agent being delivered, which may assist in selectively controlling the flow of pressurized fluid to help to prevent or minimize clogging during delivery.

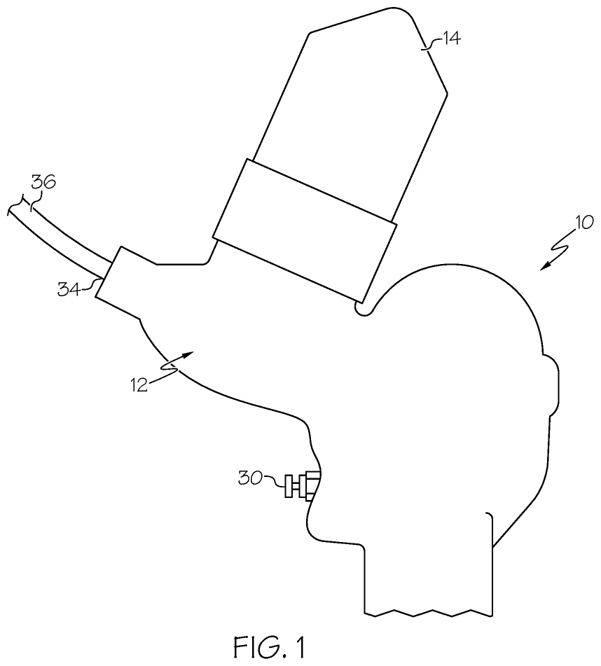

FIG. 1 shows a delivery system 10, which may be a powder delivery system. Delivery system 10 may include a handle body 12. Handle body 12 may include, or may be configured to receive, an enclosure 14 (or other source or container) storing a material (e.g., a powdered agent). Enclosure 14 may be coupled to handle body 12 for providing the agent to handle body 12, or a lid/enclosure of the agent may be screwed onto, or otherwise coupled to, enclosure 14 for supplying the agent to enclosure 14. The agent may be, for example, a powdered agent, such as a hemostatic agent. The agent may alternatively be another type of agent or material, or form of agent (e.g., a liquid or gel agent), and may have any desired function. Enclosure 14 may be removably attached to other components of delivery system 10, including components of handle body 12.

Body 12 may have a variety of features, to be discussed in further detail herein. U.S. patent application Ser. No. 16/589,633, filed Oct. 1, 2019, published as U.S. Patent Application Publication No. 2020/0100986 A1 on Apr. 2, 2022, the disclosure of which is hereby incorporated by reference in its entirety, discloses features of exemplary delivery devices and systems. The features of this disclosure may be combined with any of the features described in the above-referenced application. The features described herein may be used alone or in combination and are not mutually exclusive. Like reference numbers and/or terminology are used to denote similar structures, when possible.

Still referring to FIG. 1, delivery system 10 may include an actuation mechanism 30 used to activate flow of a pressurized fluid (e.g., gas) from a pressurized medium source in fluid communication with delivery system 10. Actuation mechanism 30 may be selectively actuated (e.g., manually depressible) or otherwise moved or actuated to control delivery of a material (e.g., a powdered agent) and pressurized fluid. The pressurized fluid alone, or a combination of a powdered agent and fluid, may be delivered from an outlet 34 of handle body 12. Outlet 34 may be in fluid communication with a catheter 36 or another component for delivering the combination of agent and fluid to a desired location within a body lumen of a patient.

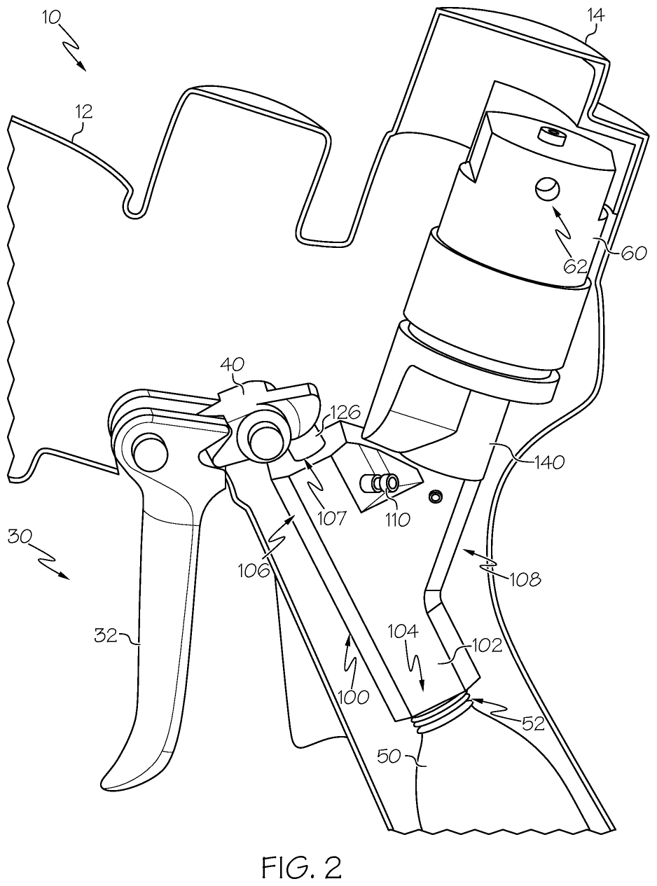

FIG. 2 shows aspects of an exemplary valve assembly 100. Valve assembly 100 may be housed within handle body 12 of delivery system 10, and selectively actuated by actuation mechanism 30. In the example, actuation mechanism 30 may include a trigger 32 that may be actuated by a user of delivery system 10, such as by manually manipulating trigger 32. Although not shown, actuation mechanism 30 may include one or more other actuation elements, such as, for example, a button, a slider, a lever, a knob, a dial, and various other suitable actuators. Delivery system 10 may include a cam 40 pivotably coupled to trigger 32 of actuation mechanism 30 and valve assembly 100. As described herein, actuation of trigger 32 may provide for a corresponding movement of cam 40 against at least a portion of valve assembly 100, thereby controlling a delivery of pressurized fluid through valve assembly 100.

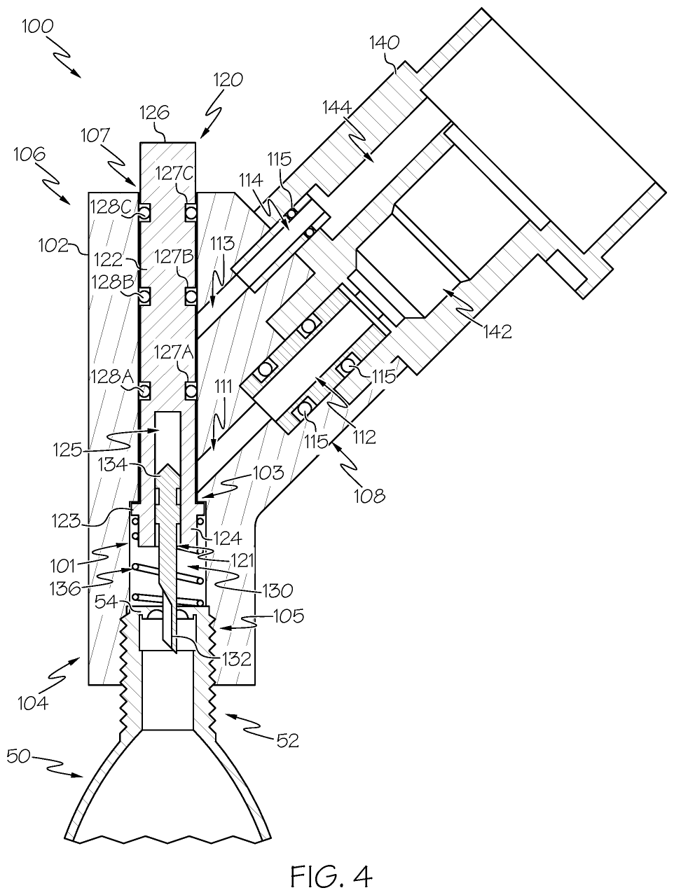

Valve assembly 100 may include a valve body 102 having a first end portion 104, a second end portion 106, and an intermediate portion 108 positioned between first end portion 104 and second end portion 106. First end portion 104 may include a first opening 105 (see FIG. 4) configured to interface with a pressurized medium source, such as, for example, a containment device 50 (e.g., a canister). In other words, first opening 105 may define an inlet for receiving the pressurized fluid from containment device 50. Second end portion 106 may include a second opening 107. Valve assembly 100 may further include a movable shuttle 120 (see FIG. 4) disposed within valve body 102, the movable shuttle 120 having a first end 124 and a second end 126 that is opposite of first end 124. In the example, at least a portion of movable shuttle 120, such as a second end 126, may extend outwardly from second end portion 106 via opening 107 to interface with cam 40. As described below, actuation of actuation mechanism 30 (e.g., via trigger 32) may provide for a release of at least a portion of the pressurized fluid from containment device 50 via a corresponding movement of movable shuttle 120 relative to valve body 102.

Still referring to FIG. 2, valve assembly 100 may include an adapter 140, and intermediate portion 108 may be configured to couple valve body 102 to adapter 140. Adapter 140 may be configured to fluidly couple a regulator 60 to valve body 102. In other embodiments, adapter 140 may be omitted entirely such that regulator 60 may be directly coupled to valve body 102, such as along intermediate portion 108. Regulator 60 may be configured to regulate an amount of the pressurized fluid released from containment device 50 at a specific pressure. For example, regulator 60 may be a dual stage regulator, or regulator 60 may include two single stage regulators, such as two piston regulators, aligned in series. Valve assembly 100 may fluidly couple containment device 50 with regulator 60 when containment device 50 is attached to adapter 140. Regulator 60 may reduce the pressure of the pressurized fluid (e.g., gas) from containment device 50 to an acceptable outlet pressure, i.e., a pressure of the gas and any material (e.g., a powdered agent) to be delivered from outlet 34.

In some embodiments, a pressure of a gas within delivery system 10, after receipt within regulator 60, may be predetermined, based on a target site (e.g., a tissue) within a patient to which the gas and agent is being dispensed. Alternatively, or additionally, the pressure of the gas controlled by regulator 60 may be determined, at least in part, on a pressure necessary to mix and/or agitate the powdered agent stored in enclosure 14. For example, regulator 60 may reduce a pressure of the pressurized fluid to approximately 50-150 pounds per square inch (“PSI”), and more particularly to approximately 20-30 PSI. The pressure of the fluid exiting regulator 60 may be approximately equal to the pressure of the fluid exiting body 12 at outlet 34 (FIG. 1). Alternatively, the pressure of the fluid at outlet 34 may be different from the pressure of the fluid exiting regulator 60. In some embodiments, dispensing system 10 may include a burst or safety valve along a fluid path downstream of regulator 60 and upstream of outlet 34, which may burst or release if a pressure of the fluid from regulator 60 is greater than a predefined threshold.

Still referring to FIG. 2, regulator 60 may include an opening 62 that is configured to vent the pressurized fluid received therein from valve assembly 100. Stated differently, opening 62 may be in communication with an atmospheric pressure within body 12 to facilitate pressure control of the fluid released from containment device 50. In some embodiments, the fluid may be released into atmospheric pressure within handle body 12. In other embodiments, handle body 12 may include an opening and/or a fluid connection extending to an exterior of handle body 12 for releasing the pressurized fluid.

Valve assembly 100 may include an outlet port 110 on valve body 102, such as along intermediate portion 108. Although not shown, it should be appreciated that a tube and/or other suitable device may be coupled to outlet port 110 for connecting outlet port 110 to one or more components of delivery system 10 that are in fluid communication with outlet 34. Accordingly, outlet port 110 may be in fluid communication with outlet 34 to facilitate delivery of the pressurized fluid received from regulator 60 to catheter 36 via the tube.

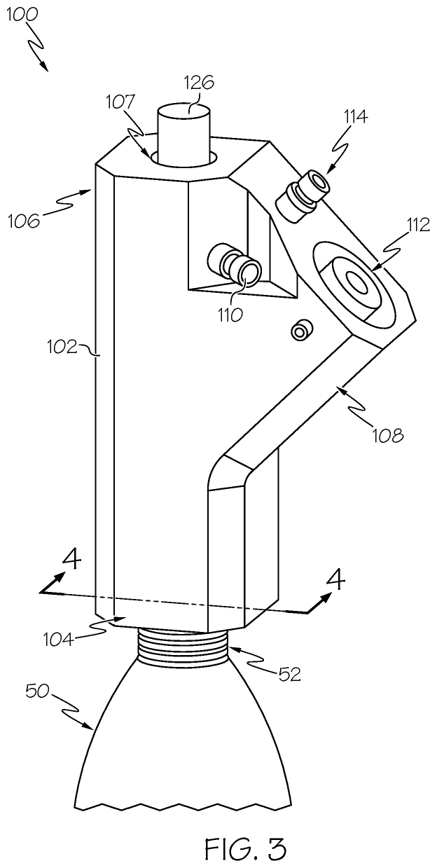

Referring now to FIG. 3, intermediate portion 108 may include an inlet connector 112 and an outlet connector 114. In some embodiments, one or more of connectors 112, 114 may be coupled to respective portions of valve body 102, for example, to intermediate portion 108, while in other embodiments connectors 112, 114 may be integral with valve body 102, for example, with intermediate portion 108. Each of inlet connector 112 and outlet connector 114 may be in fluid communication with a respective internal conduit of valve body 102 (see FIG. 4). Further, each of inlet connector 112 and outlet connector 114 may be configured to fluidly couple the respective internal conduits of valve body 102 with corresponding channels of adapter 140, such that valve body 102 may be in fluid communication with regulator 60 via inlet connector 112 and outlet connector 114 when regulator 60 is coupled to adapter 140.

As best seen in FIG. 4, each of inlet connector 112 and outlet connector 114 may include one or more gaskets 115 (e.g., seals, O-rings, etc.) to form a fluid-tight seal between adapter 140 and valve body 102. Valve body 102 may include an inner channel 101 extending between first end portion 104 and second end portions 106. In the example, inner channel 101 may have a longitudinal length extending between first opening 105 at first end portion 104 and second opening 107 at second end portion 106. Valve body 102 may further include a first (outlet) conduit 111 and a second (inlet) conduit 113 extending from inner channel 101 through intermediate portion 108. Stated differently, first (outlet) conduit 111 and second (inlet) conduit 113 may be in fluid communication with inner channel 101.

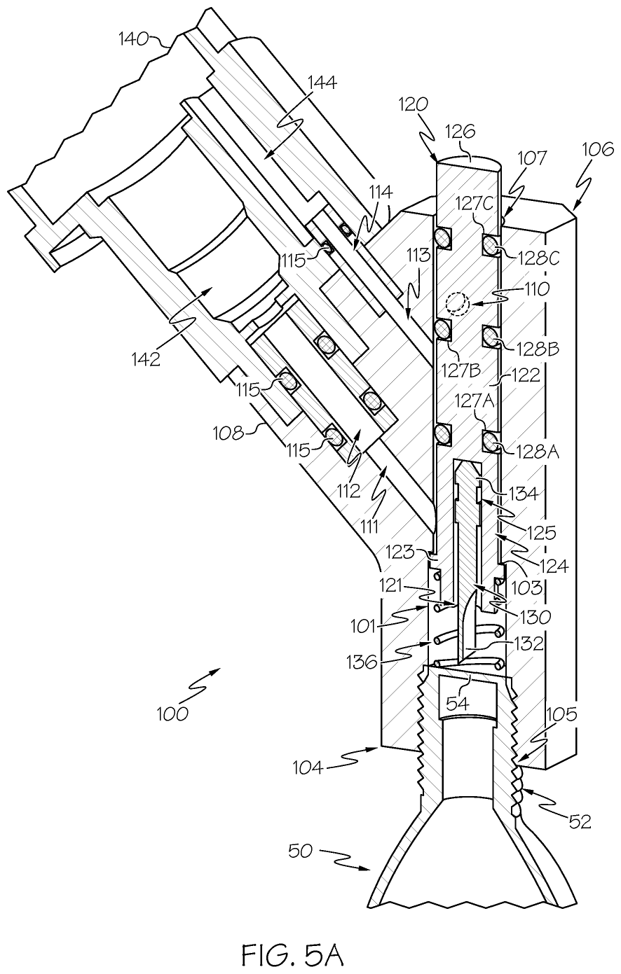

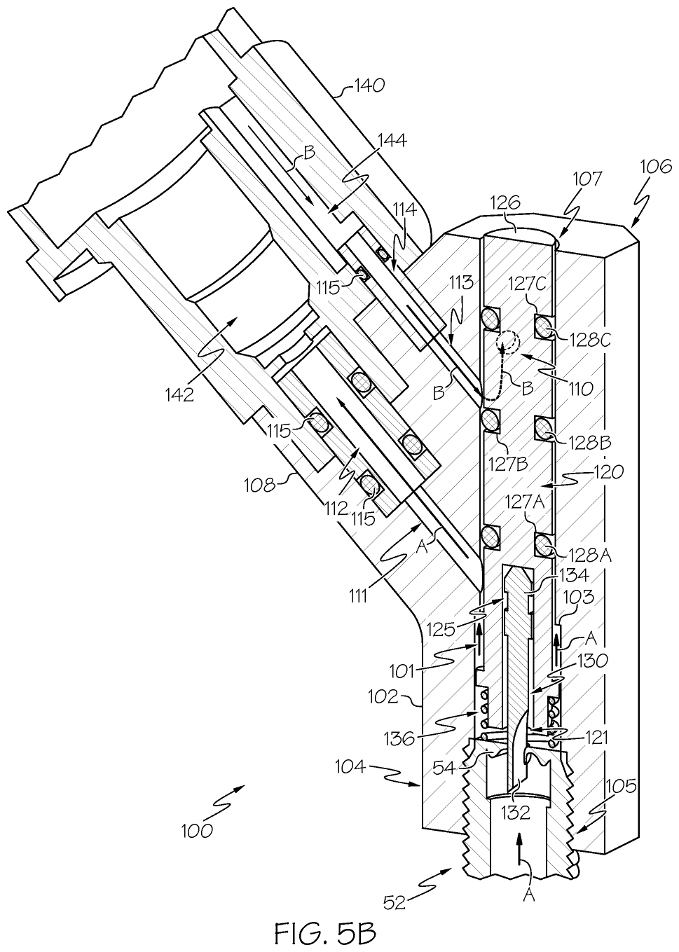

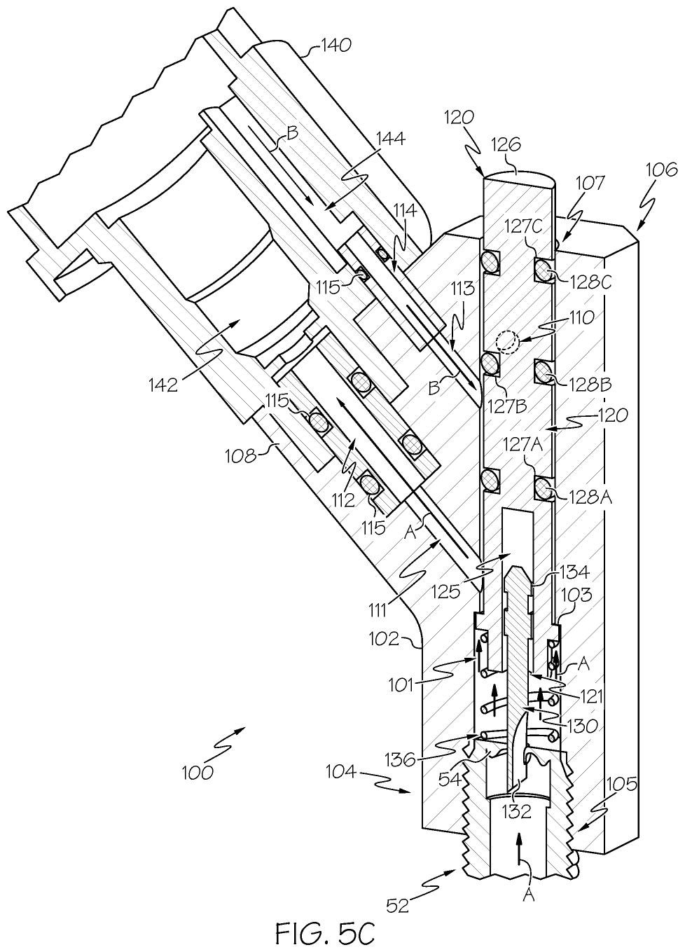

First (outlet) conduit 111 may provide an outlet from inner channel 101, and may be fluidly coupled to an inlet channel 142 of adapter 140 via inlet connector 112. Second (inlet) conduit 113 may provide an inlet into inner channel 101, and may be fluidly coupled to an outlet channel 144 of adapter 140 via outlet connector 114. Each of first (outlet) conduit 111 and second (inlet) conduit 113 may be angled (e.g., transverse) relative to a central longitudinal axis of inner channel 101, which may be parallel to a longitudinal length of valve body 102. As described in detail below and as shown in FIG. 5B, a high pressure fluid A received in valve assembly 100 (e.g., from containment device 50) may be directed into regulator 60 via a first fluid path defined at least partially by inner channel 101, first (outlet) conduit 111, inlet connector 112, and inlet channel 142. Further, as shown in FIGS. 5B and 5C, a low pressure fluid B (converted/adjusted from and relative to the high pressure fluid A) exiting regulator 60 may be directed into valve assembly 100 via a second fluid path defined at least partially by outlet channel 144, outlet connector 114, second (inlet) conduit 113, and inner channel 101.

Still referring to FIG. 4, inner channel 101 may be at least partially defined by a ledge 103 extending radially-inward toward a central longitudinal axis of inner channel 101 (extending parallel to the longitudinal length of valve body 102). As described herein, ledge 103 may be sized, shaped, and/or otherwise configured to engage at least a portion of movable shuttle 120 within inner channel 101, such as a lateral projection or flange 123. In the example, ledge 103 may be positioned relatively closer to first end portion 104 than to second end portion 106, and inner channel 101 may have a diameter that varies along a longitudinal length of valve body 102. For example, inner channel 101 may have a first diameter along a portion extending between ledge 103 and first end portion 104 that is greater than a second diameter along another portion extending between ledge 103 and second end portion 106. Accordingly, ledge 103 may define an interface within valve body 102 at which the first diameter of inner channel 101 transitions to the second diameter.

As described in detail herein, inner channel 101 may be sized, shaped, and/or otherwise configured to receive a biasing mechanism 136 of valve assembly 100. For example, inner channel 101 may receive biasing mechanism 136 within the portion of inner channel 101 having the greater, first diameter between ledge 103 and first end portion 104. As shown in FIG. 4, biasing mechanism 136 may be a spiral spring. First opening 105 may include a threaded portion along an inner surface defining first opening 105. The threaded portion may be configured to mesh with or otherwise be coupled to a corresponding threaded portion along an outer surface of a neck 52 of containment device 50 to help facilitate a connection between valve body 102 and containment device 50. Accordingly, containment device 50 may be rotatably coupled to valve assembly 100 at first end portion 104.

Still referring to FIG. 4, movable shuttle 120 may be slidably received within inner channel 101 of valve body 102. Movable shuttle 120 may include a shuttle body 122 having a longitudinal length defined between a first end 124 and a second end 126 that is opposite of the first end 124. Movable shuttle 120 may be sized and shaped in accordance with a cross-sectional dimension of inner channel 101. In the example, shuttle body 122 may have a generally cylindrical shape that has a diameter that is less than the diameter of inner channel 101. Accordingly, an exterior surface of shuttle body 122 may be offset and/or separated from contacting an interior surface of valve body 102 defining inner channel 101. As such, movable shuttle 120 may be configured to move (e.g., translate, rotate, etc.) within inner channel 101 and relative to valve body 102. As described herein, movable shuttle 120 may be operable to move between a plurality of positions relative to valve body 102 to transition valve assembly 100 between a corresponding plurality of configurations.

Movable shuttle 120 may include one or more recesses and/or cavities formed along the longitudinal length of shuttle body 122 for receiving at least one gasket (e.g., a seal, O-ring, etc.). In the example, movable shuttle 120 may include three recesses along shuttle body 122 between first end 124 and second end 126, with a corresponding first gasket 128A, second gasket 1288, and third gasket 128C received within each recess. First gasket 128A may be positioned in a first recess 127A along shuttle body 122 proximate to first end 124 relative to second gasket 1288 and third gasket 128C. Second gasket 128B may be positioned in a second recess 127B along shuttle body 122 between first gasket 128A and third gasket 128C. Third gasket 128C may be positioned in a third recess 127C along shuttle body 122 proximate to second end 126 relative to first gasket 128A and second gasket 1288. It should be appreciated that valve assembly 100 may include additional and/or fewer gaskets and corresponding recesses without departing from a scope of this disclosure.

As seen in FIG. 4, each of first gasket 128A, second gasket 1288, and third gasket 128C may be sized and/or shaped to have a cross-sectional dimension that extends radially-outward from the corresponding recess 127A, 127B, 127C on shuttle body 122, such that the gaskets 128A, 1288, 128C may be configured to interface with the inner surface of valve body 102 defining inner channel 101. In other words, gaskets 128A, 1288, 128C may be sized to within, and are at least partially greater than, the corresponding recesses 127A, 1278, 127C such that at least a portion of each gasket 128A, 1288, 128C may extend out of the respective recess 127A, 1278, 127C. As described herein, the gaskets 128A, 1288, 128C may be configured to abut against the inner surface of valve body 102, such as an inner wall of inner channel 101, to form a fluid seal at three respective locations within inner channel 101. Valve assembly 100 may be configured to control a fluid flow path of the pressurized fluid released from containment device 50 and received within valve body 102 in response to a corresponding position of movable shuttle 120, and particularly the gaskets 128A, 1288, 128C, relative to inner channel 101.

Movable shuttle 120 may include flange 123 proximate to first end 124 relative to second end 126. Flange 123 may be sized and shaped with a diameter that is less than a first diameter of inner channel 101 between ledge 103 and first end portion 104. Accordingly, flange 123 may be offset and/or separated from contacting the interior surface of valve body 102 defining inner channel 101. Further, the diameter of flange 123 may be greater than a second diameter of inner channel 101 between ledge 103 and second end portion 106. Accordingly, flange 123 may abut against and contact ledge 103 within inner channel 101 when movable shuttle 120 moves in an upward direction in FIG. 4 toward second end portion 106.

As such, valve body 102 may be configured to restrict movement of movable shuttle 120 within inner channel 101 and relative to valve body 102 when flange 123 engages ledge 103. Biasing mechanism 136 may be received within a portion of inner channel 101 between ledge 103 and first end portion 104. Biasing mechanism 136 may be disposed about a portion of shuttle body 122, such as along first end 124, with one end of biasing mechanism 136 positioned against flange 123. Biasing mechanism 136 may be configured to bias movable shuttle 120 in an upward direction in FIG. 4 relative to valve body 102. For example, biasing mechanism 136 may be configured to apply an upward force against flange 123, thereby biasing movable shuttle 120 upward.

Still referring to FIG. 4, movable shuttle 120 may include a closed channel 125 within shuttle body 122. In the example, closed channel 125 may extend from an opening 121 at first end 124 and toward second end 126. Closed channel 125 may terminate within shuttle body 122, such as, for example, at a location proximate to a location of first recess 127A. A size, shape, and longitudinal length of closed channel 125 may correspond to a size, shape, and length of a piercing element 130 of valve assembly 100 received therein.

Piercing element 130 may have a longitudinal length defined between a front end 132 and a rear end 134. Piercing element 130 may be configured to move within closed channel 125 and relative to shuttle body 122 of movable shuttle 120, such as, for example, between one or more of a plurality of positions. Piercing element 130 may be configured to move within inner channel 101 and relative to valve body 102, such as with movable shuttle 120. As described herein, in at least a first position relative to shuttle body 122, piercing element 130 may move simultaneously with movable shuttle 120, while in at least a second position relative to shuttle body 122, piercing element 130 may move independent of movable shuttle 120.

In some examples, piercing element 130, for example, a portion of front end 132, may include a lance and/or a needle. Front end 132 of piercing element 130 may define a sharp tip that is configured to pierce one or more components of delivery system 10 (FIG. 1), such as, for example, a seal 54 of containment device 50. Rear end 134 of piercing element 130 may define a bulbous tip that is configured to interface with an inner surface of shuttle body 122 defining closed channel 125, such that rear end 134 may interface with closed channel 125 to form a frictional resistance within shuttle body 122 to maintain piercing element 130 within movable shuttle 120.

Still referring to FIG. 4, biasing mechanism 136 may abut against a bottom surface of flange 123 and a top surface of the neck 52 of containment device 50 when containment device 50 is coupled to valve body 102. Absent an actuation of actuation mechanism 30, biasing mechanism 136 may bias (e.g., apply a force) movable shuttle 120 relatively upward through inner channel 101 to a position away from first end portion 104 and toward second end portion 106. As described below, biasing mechanism 136 may transition from a first (expanded) configuration to a second (compressed) configuration in response to an actuation of actuation mechanism 30, and return from the second configuration to the first configuration upon release of actuation mechanism 30.

FIGS. 5A-5C illustrate an exemplary use of delivery system 10, with valve assembly 100 in a first configuration, movable shuttle 120 in a first position relative to valve body 102, and piercing element 130 in a first position relative to shuttle body 122, as depicted in FIG. 5A. For example, absent an application of a downward (longitudinal) force onto movable shuttle 120 (e.g., by cam 40 (FIG. 2) against second end 126 from an actuation of actuation mechanism 30), an upward (longitudinal) force applied against first end 124 by biasing mechanism 136 may bias movable shuttle 120 upward to the first position relative to valve body 102. In this instance, biasing mechanism 136 may be in the first (expanded) configuration, and may maintain shuttle body 122 at the first position until an opposite, downward force that is greater than the upward force of biasing mechanism 136 is applied to movable shuttle 120 (e.g., by cam 40).

In the first position of movable shuttle 120, shuttle body 122 may be positioned at an upward-most extent relative to valve body 102 such that flange 123 may be engaged against ledge 103 within inner channel 101. In this position, second end 126 of movable shuttle 120 may extend outwardly from second end portion 106 of valve body 102 (via second opening 107) to its upward-most extent. When in the first configuration of valve assembly 100, first gasket 128A may be positioned between first (outlet) conduit 111 and second (inlet) conduit 113, second gasket 128B may be positioned between second (inlet) conduit 113 and outlet port 110, and third gasket 128C may be positioned between outlet port 110 and second opening 107. Accordingly, second gasket 128B may be positioned to inhibit fluid communication between second (inlet) conduit 113 and outlet port 110. Although not shown, cam 40 (FIG. 2) may abut against second end 126 when movable shuttle 120 is in the first position.

In the first position of piercing element 130, piercing element 130 may be positioned at an upward-most extent relative to shuttle body 122 such that rear end 134 may abut against a terminal end of closed channel 125. In this position, piercing element 130 may be received within shuttle body 122 with front end 132 extending at least partially outwardly from closed channel 125 via opening 121. In other words, front end 132 may be exposed from shuttle body 122 when piercing element 130 is in the first position. Front end 132 may be spaced apart from seal 54 when movable shuttle 120 is in the first position and piercing element 130 is in the first position. Movement of movable shuttle 120 within inner channel 101 may cause front end 132 to contact seal 54 of containment device 50. In the first configuration of valve assembly 100, seal 54 of containment device 50 may remain intact such that the pressurized fluid stored therein may be maintained within containment device 50.

Referring now to FIG. 5B, valve assembly 100 may transition to a second configuration upon actuation of actuation mechanism 30 and movement of cam 40. In this instance, movable shuttle 120 may move to a second position relative to valve body 102 and piercing element 130 may remain in the first position relative to shuttle body 122. For example, actuation of actuation mechanism 30 may move cam 40 (FIG. 2) into engagement with second end 126, thereby applying a downward (longitudinal) force onto movable shuttle 120 that is greater than the opposite, upward force applied against first end 124 by biasing mechanism 136. In this instance, biasing mechanism 136 may transition to the second (compressed) configuration as movable shuttle 120 moves downward relative to valve body 102 to the second position.

In the second position of movable shuttle 120, shuttle body 122 may be positioned at a downward-most extent relative to valve body 102 such that flange 123 may be disengaged from ledge 103. In this position, second end 126 of movable shuttle 120 may extend through second end portion 106 of valve body 102 (via second opening 107) to its downward-most extent. When in the second configuration of valve assembly 100, first gasket 128A and second gasket 128B may be positioned between first (outlet) conduit 111 and second (inlet) conduit 113, and third gasket 128C may be positioned between outlet port 110 and second opening 107. Accordingly, second gasket 128B may be positioned to allow fluid communication between second (inlet) conduit 113 and outlet port 110.

Still referring to FIG. 5B, in the first position of piercing element 130, piercing element 130 may remain positioned at the upward-most extent relative to shuttle body 122 such that rear end 134 continues to abut against the terminal end of closed channel 125. In this position, piercing element 130 is maintained within shuttle body 122 with front end 132 extending at least partially outward from closed channel 125 via opening 121. With front end 132 exposed from shuttle body 122, movement of movable shuttle 120 within inner channel 101 may cause front end 132 to contact seal 54 of containment device 50, thereby puncturing seal 54. Accordingly, in the second configuration of valve assembly 100, seal 54 of containment device 50 may be permanently deformed such that the high pressure fluid A stored therein may be released into valve body 102 and received within inner channel 101. In the second position, first end 124 may be spaced apart from seal 54 to allow the high pressure fluid A to exit containment device 50 and enter inner channel 101. Seal 54 may include a deformable barrier positioned over an opening of containment device 50 at a terminal end of neck 52. Seal 54 may be at least partially flexible, and configured to be pierced and/or punctured upon encountering a sharp object, such as front end 132 of piercing element 130.

A high pressure fluid A released from containment device 50 may travel along a first fluid path through inner channel 101 until encountering first gasket 128A positioned relatively downstream (i.e., above in FIG. 5B) first (outlet) conduit 111. First gasket 128A may be configured to inhibit the high pressure fluid A from flowing (upward in FIG. 5B) beyond a location of first gasket 128A relative to inner channel 101. Accordingly, first gasket 128A may redirect the high pressure fluid A into first (outlet) conduit 111 and into regulator 60 via inlet channel 142 of adapter 140. After passing through regulator 60, a low pressure fluid B may exit regulator 60 (coupled to adapter 140) and travel along a second fluid path through outlet channel 144 of adapter 140 and second (inlet) conduit 113 until entering inner channel 101.

Still referring to FIG. 5B, with movable shuttle 120 at the second position, and second gasket 128B and third gasket 128C positioned on opposing sides of second (inlet) conduit 113, the low pressure fluid B may enter inner channel 101 between second gasket 128B and third gasket 128C. Accordingly, second gasket 128B may be configured to inhibit the low pressure fluid B from flowing (downward) beyond a location of second gasket 128B relative to inner channel 101, and third gasket 128C may be configured to inhibit the low pressure fluid B from flowing (upward in FIG. 5B) beyond a location of third gasket 128B relative to inner channel 101. With outlet port 110 positioned between second gasket 128B and third gasket 128B, the pair of gaskets 128B, 128C may cooperatively redirect the low pressure fluid B into outlet port 110 for delivery toward catheter 36 (FIG. 1).

Referring now to FIG. 5C, valve assembly 100 may transition to a third configuration upon releasing actuation mechanism 30. In this instance, movable shuttle 120 may move to a third position relative to valve body 102 and piercing element 130 may move to a second position relative to shuttle body 122. In some examples, the third position of movable shuttle 120 may be substantially similar to the first position (FIG. 5A), such that releasing actuation mechanism 30 may return movable shuttle 120 to the first position. For example, ceasing actuation of actuation mechanism 30 may move cam 40 away from movable shuttle 120, thereby disengaging second end 126. In this instance, biasing mechanism 136 may be returned to the first (expanded) configuration in which the upward (longitudinal) force applied against first end 124 by biasing mechanism 136 may cause movable shuttle 120 to move (upward) relative to valve body 102 and through inner channel 101.

In the third position of movable shuttle 120, shuttle body 122 may be repositioned at the upward-most extent relative to valve body 102 such that flange 123 may be engaged with ledge 103. In this position, second end 126 of movable shuttle 120 may extend through second end portion 106 of valve body 102 (via second opening 107) to its upward-most extent. When in the third configuration of valve assembly 100, first gasket 128A may remain between first (outlet) conduit 111 and second (inlet) conduit 113, second gasket 1288 may be repositioned between second (inlet) conduit 113 and outlet port 110, and third gasket 128C may remain between outlet port 110 and second opening 107. Accordingly, second gasket 1288 may be positioned to inhibit fluid communication between second (inlet) conduit 113 and outlet port 110.

Still referring to FIG. 5C, in the second position of piercing element 130, piercing element 130 may be positioned at a downward-most extent relative to shuttle body 122 such that rear end 134 is offset and/or separated from the terminal end of closed channel 125. In this position, piercing element 130 is at least partially maintained within shuttle body 122 with front end 132 extending outward from closed channel 125 at first end 124. With piercing element 130 independently moveable relative to movable shuttle 120, front end 132 may remain in contact with seal 54 as movable shuttle 120 moves within inner channel 101 from the second position to the third position. Accordingly, piercing element 130 may be configured to maintain seal 54 in an opened state.

It should be appreciated that a failure of dispensing system 10, such as caused by movable shuttle 120 being stuck in the second (downward-most) position due to an engagement between front end 132 and seal 54, may be minimized due to the independent movement of movable shuttle 120 and piercing element 130. Accordingly, valve assembly 100 may help to inhibit an inadvertent release of the low pressure fluid B from dispensing system 10, as caused by movable shuttle 120 being stuck in the second position when actuation mechanism 30 is in an unactuated state.

Accordingly, in the third configuration of valve assembly 100, front end 132 may continue to extend through seal 54 such that the high pressure fluid A stored within containment device 50 may continue to be released into valve body 102 and received within inner channel 101. Stated differently, valve body 102 may remain in fluid communication with containment device 50 when actuation mechanism 30 is released. It should be appreciated that, in instances where piercing element 130 moves away (upward in FIGS. 5A-5C) from containment device 50 in response to an upward movement of movable shuttle 120, seal 54 may remain permanently deformed such that the high pressure fluid A may continue to be released despite a retraction of front end 132 from seal 54.

The high pressure fluid A released from containment device 50 may travel along the first fluid path through inner channel 101 until encountering first gasket 128A positioned relatively above first (outlet) conduit 111. First gasket 128A may be configured to inhibit the high pressure fluid A from flowing (upward in FIGS. 5A-5C) beyond a location of first gasket 128A relative to inner channel 101. Accordingly, first gasket 128A may redirect the high pressure fluid A into first (outlet) conduit 111 and into regulator 60 via inlet channel 142 of adapter 140. Upon reducing the pressure of the high pressure fluid A, the low pressure fluid B may exit regulator 60 and travel along the second fluid path through outlet channel 144 of adapter 140 and second (inlet) conduit 113 until entering inner channel 101.

Still referring to FIG. 5C, with movable shuttle 120 at the third position, second gasket 128B may be positioned between second (inlet) conduit 113 and outlet port 110, and third gasket 128C may remain between outlet port 110 and second opening 107. Accordingly, second gasket 128B may be positioned to inhibit fluid communication between second (inlet) conduit 113 and outlet port 110. Accordingly, the low pressure fluid B may enter inner channel 101 between first gasket 128A and second gasket 128B. First gasket 128A may be configured to inhibit the low pressure fluid B from flowing (downward in FIG. 5C) beyond a location of first gasket 128B relative to inner channel 101, and second gasket 128B may be configured to inhibit the low pressure fluid B from flowing (upward) beyond a location of second gasket 128B relative to inner channel 101. With outlet port 110 positioned above second gasket 128B, second gasket 128B may fluidly seal outlet port 110 to help inhibit delivery of the low pressure fluid B toward catheter 36.

It should be appreciated that valve assembly 100 may be operable to provide consistent control and operation of dispensing system 10 by requiring a substantially similar force to actuate actuation mechanism 30 irrespective of a current configuration of valve assembly 100 (e.g., the first configuration of FIG. 5A, the second configuration of FIG. 5B, and the third configuration of FIG. C). Irrespective of an accumulation of the high pressure fluid A and/or the low pressure fluid B within one or more portions of valve body 102, such as in inner channel 101, valve assembly 100 may mitigate and/or minimize an opposing force applied against movable shuttle 120 (e.g., by the high pressure fluid A and/or the low pressure fluid B in an upward direction away from containment device 50) such that a force required to translate movable shuttle 120 (e.g., in a downward direction toward containment device 50) and dispense a pressurized fluid to outlet port 110 may be substantially constant.

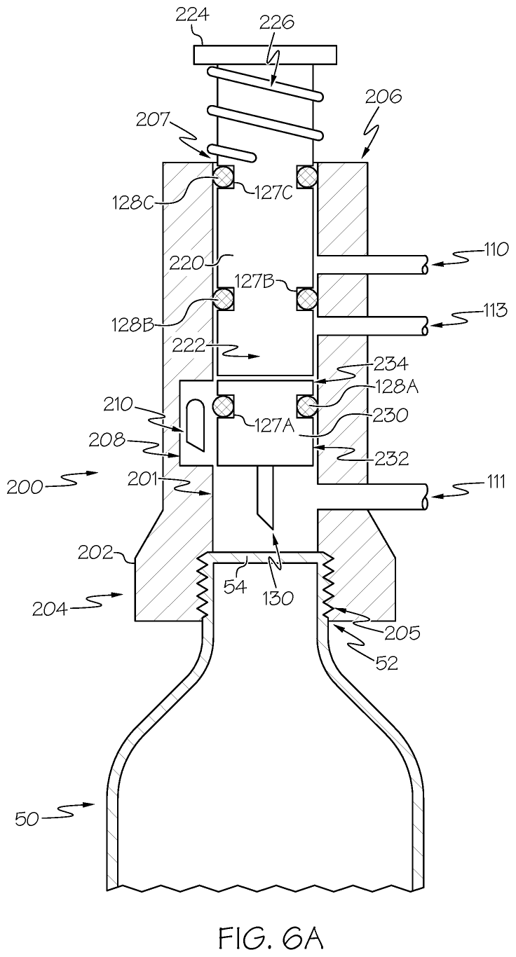

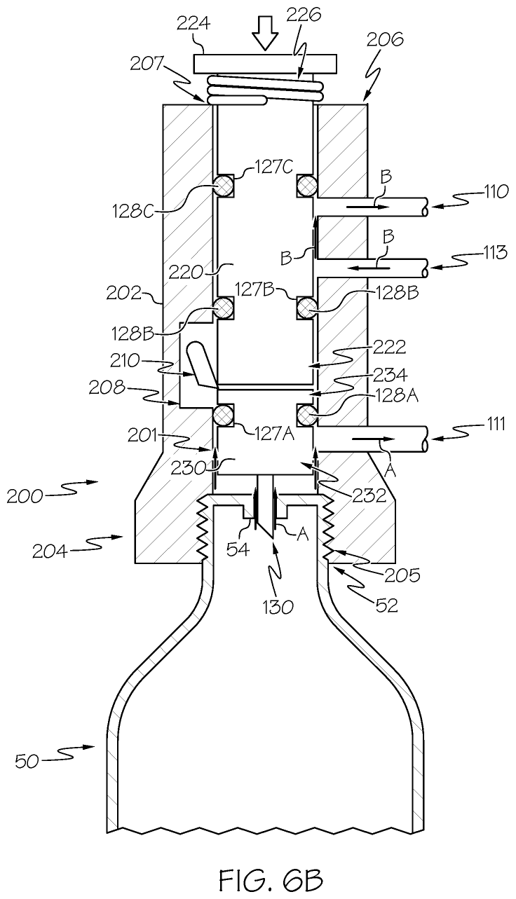

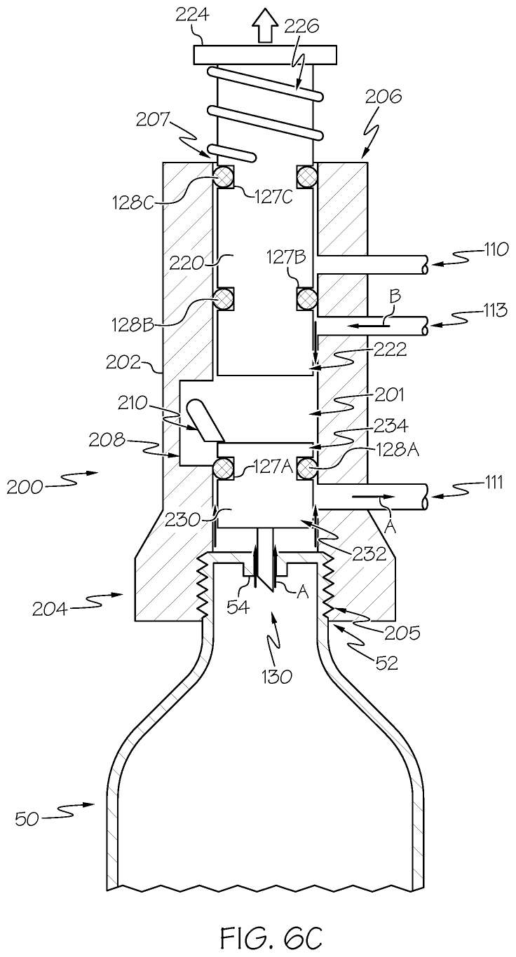

Referring now to FIGS. 6A-6C, aspects of another exemplary valve assembly 200 are shown. Valve assembly 200 may be configured similar to valve assembly 100 except for the differences explicitly described herein, such that like reference numerals are used to identify like components. Valve assembly 200 may be housed within body 12 of delivery system 10, and selectively actuated by actuation mechanism 30 (see FIG. 1).

Referring specifically to FIG. 6A, valve assembly 200 may include a valve body 202 having a first end portion 204 and a second end portion 206. First end portion 204 may include a first opening 205 configured to interface with a pressurized medium source, such as, containment device 50. For example, first opening 205 may include a threaded portion along an inner surface defining first opening 205 to mesh with or otherwise couple to the threaded portion of neck 52. Accordingly, containment device 50 may be rotatably coupled to valve assembly 200 at first end portion 204. Second end portion 206 may include a second opening 207.

Valve body 202 may include an inner channel 201 extending between first end portion 204 and second end portion 206, along with outlet port 110, first (outlet) conduit 111, and second (inlet) conduit 113 extending from inner channel 101. Accordingly, outlet port 110, first (outlet) conduit 111, and second (inlet) conduit 113 may be in fluid communication with inner channel 201. Valve body 202 may further include a cavity 208 positioned adjacent to inner channel 101, with cavity 208 being sized, shaped, and/or otherwise configured to receive a locking mechanism 210. Locking mechanism 210 may include a tab, a pawl, a beam, and/or a projection that is movably coupled within valve body 202, such as about a pin. Locking mechanism 210 may be configured to move within cavity 208 from a retracted position (FIG. 6A) to an extended configuration (FIG. 6C).

In some embodiments, locking mechanism 210 may be biased toward the extended configuration, such as, for example, by a biasing mechanism coupled thereto. In other embodiments, locking mechanism 210 may be selectively movable between the retracted and extended configurations. As described in detail herein, locking mechanism 210 may be configured to extend out of cavity 208 and into inner channel 201 to engage one or more components of valve assembly 200.

Still referring to FIG. 6A, valve assembly 200 may further include a first movable shuttle 220 and a second movable shuttle 230 disposed within valve body 202. In the example, first movable shuttle 220 may have a longitudinal length defined between a first end 222 and a second end 224, and second movable shuttle 230 may have a longitudinal length defined between a first (lower) end 232 and a second (upper) end 234. Each of first movable shuttle 220 and second movable shuttle 230 may be sized, shaped, and/or otherwise configured to be received within inner channel 201. In the example, first movable shuttle 220 may be positioned within inner channel 201 relatively above second movable shuttle 230, such that first movable shuttle 220 may be positioned proximate to second end portion 206 (relative to second movable shuttle 230) and second movable shuttle 230 may be positioned proximate to first end portion 204 (relative to first movable shuttle 220).

First end 222 of first movable shuttle 220 may be configured to interface with second end 234 of second movable shuttle 230, and second end 224 of first movable shuttle 220 may extend outwardly from second end portion 206 via opening 207 to interface with cam 40 (FIG. 2). First end 232 of second movable shuttle 230 may be coupled to piercing element 130, and second end 234 of second movable shuttle 230 may be configured to interface with first end 222 of first movable shuttle 220. In the example, piercing element 130 may be fixed relative to second movable shuttle 230. Valve assembly 200 may further include a biasing mechanism 226 disposed about at least a portion of first movable shuttle 220. In the example, biasing mechanism 226 may be coupled to first movable shuttle 220 proximate to second end 224, and positioned external to inner channel 201.

Still referring to FIG. 6A, one end of biasing mechanism 226 may be positioned against second end portion 206 of valve body 202, and an opposite end of biasing mechanism 236 may be positioned against second end 224. In the example, second end 224 may have a greater cross-sectional dimension and/or define a widened portion relative to first movable shuttle 220. Accordingly, biasing mechanism 226 may be configured to abut against the widened portion of second 224. Biasing mechanism 236 may be configured to bias the first movable shuttle 220 in an upward direction relative to valve body 202, in response to applying an upward force against second end 224. It should be appreciated that first movable shuttle 220 and second movable shuttle 230 may be coupled to one another, along an interface between first end 222 and second end 234, when valve assembly 200 is in a first configuration shown in FIG. 6A.

In some embodiments, first movable shuttle 220 and second movable shuttle 230 may be coupled to one another via a frictional engagement with one another and/or with the interior surface defining inner channel 201. In other embodiments, first movable shuttle 220 and second movable shuttle 230 may be coupled to one another with an adhesive positioned along the interface between first end 222 and second end 234. It should be appreciated that first movable shuttle 220 and second movable shuttle 230 may be coupled to one another by various other suitable means without departing from a scope of this disclosure.

First movable shuttle 220 and second movable shuttle 230 may include one or more recesses and/or cavities formed along the respective longitudinal lengths of first movable shuttle 220 and second movable shuttle 230 for receiving at least one gasket (e.g., a seal, O-ring, etc.). In the example, second movable shuttle 230 may include first recess 127A between first end 232 and second end 234, with a corresponding first gasket 128A received within first recess 127A. First movable shuttle 220 may include second recess 127B and third recess 127 between first end 222 and second end 224, with a corresponding second gasket 128B and third gasket 128C received within each respective recess.

In exemplary use, valve assembly 200 may be in a first configuration with first movable shuttle 220 and second movable shuttle 230 each in a respective first position relative to valve body 202, as depicted in FIG. 6A. Absent a downward (longitudinal) force applied onto first movable shuttle 220 (e.g., by cam 40 against second end 224), an upward (longitudinal) force applied against second end 224 by biasing mechanism 236 may move first movable shuttle 220 and second movable shuttle 230 upward to the first position relative to valve body 202. In this instance, biasing mechanism 236 may be in a first (expanded) configuration, and may maintain first movable shuttle 220 and second movable shuttle 230 at the first position until an opposite, downward force that is greater than the upward force of biasing mechanism 236 is applied thereto.

In the first position, each of first movable shuttle 220 and second movable shuttle 230 may be positioned at an upward-most extent relative to valve body 202. In this position, second end 224 may extend outwardly from second end portion 206 of valve body 202 (via second opening 207) to its upward-most extent. In the first configuration of valve assembly 200, first gasket 128A may be positioned between first (outlet) conduit 111 and second (inlet) conduit 113, second gasket 1288 may be positioned between second (inlet) conduit 113 and outlet port 110, and third gasket 128C may be positioned between outlet port 110 and second opening 207. Accordingly, second gasket 1288 may be positioned to inhibit fluid communication between second (inlet) conduit 113 and outlet port 110.

Still referring to FIG. 6A, in the first position of second movable shuttle 230, piercing element 130 may be positioned at an upward-most extent relative to valve body 202. In this position, piercing element 130 may be offset and/or separated from contacting seal 54. With piercing element 130 coupled to first end 232 of second movable shuttle 230, movement of the movable shuttle, and specifically second movable shuttle 230, may cause piercing element 130 to contact seal 54. In the first configuration of valve assembly 200, seal 54 of containment device 50 may remain intact such that the pressurized fluid stored therein may be maintained within containment device 50.

When in the first configuration of valve assembly 200, second movable shuttle 230 may be in the first position in which locking mechanism 210 is inhibited from extending radially-outward from cavity 208 and inward into inner channel 201 by second movable shuttle 230. Stated differently, second movable shuttle 230 may overlap with cavity 208 when in the first position such that locking mechanism 210 is maintained in a retracted positioned within cavity 208, thereby allowing movement of second movable shuttle 230 through inner channel 201.

Referring now to FIG. 6B, valve assembly 200 (e.g., first movable shuttle 220 and second movable shuttle 230) may transition to a second configuration upon actuation of actuation mechanism 30. In this instance, first movable shuttle 220 and second movable shuttle 230 may each move to a respective second position relative to valve body 202. For example, actuation of actuation mechanism 30 may move cam 40 into engagement with second end 224, thereby applying a downward (longitudinal) force onto first movable shuttle 220 that is greater than the opposite, upward force applied against second end 224 by biasing mechanism 236. In this instance, biasing mechanism 236 may be transitioned to a second (compressed) configuration as first movable shuttle 220 and second movable shuttle 230 moves downward relative to valve body 202 to the second position.

In the second position, each of first movable shuttle 220 and second movable shuttle 230 may be positioned at a downward-most extent relative to valve body 202. When in the second configuration of valve assembly 200, first gasket 128A and second gasket 128B may be positioned between first (outlet) conduit 111 and second (inlet) conduit 113, and third gasket 128C may be positioned between outlet port 110 and second opening 207. Accordingly, second gasket 128B may be positioned to allow fluid communication between second (inlet) conduit 113 and outlet port 110.

Still referring to FIG. 6B, in the second position of second movable shuttle 230, piercing element 130 may simultaneously move to a downward-most extent relative to valve body 202 such that piercing element 130 contacts and punctures seal 54. Accordingly, in the second configuration of valve assembly 200, seal 54 of containment device 50 may be permanently deformed such that the high pressure fluid A stored therein may be released into valve body 202 and received within inner channel 201. It should be appreciated that the high pressure fluid A may push against the movable shuttle upon its release from containment device 50. By way of illustrative example, the high pressure fluid A may range from about 800 PSI to 1000 PSI, such a corresponding force of about 75 pounds (lbs.) to 90 lbs. may act against second movable shuttle 230 and first movable shuttle 220.

The high pressure fluid A released from containment device 50 may travel along a first fluid path through inner channel 201 until encountering first gasket 128A positioned relatively above first (outlet) conduit 111. First gasket 128A may be configured to help inhibit the high pressure fluid A from flowing (upward) beyond a location of first gasket 128A relative to inner channel 201. Accordingly, first gasket 128A may redirect the high pressure fluid A into first (outlet) conduit 111 and into regulator 60. Upon reducing the pressure of the high pressure fluid A, a low pressure fluid B may exit regulator 60 and travel along a second fluid path through second (inlet) conduit 113 until entering inner channel 201.

Still referring to FIG. 6B, with first movable shuttle 220 and second movable shuttle 230 at the second position, and second gasket 128B and third gasket 128C positioned on opposing sides of second (inlet) conduit 113, the low pressure fluid B may enter inner channel 201 between second gasket 128B and third gasket 128C. Accordingly, second gasket 128B may be configured to inhibit the low pressure fluid B from flowing (downward) beyond a location of second gasket 128B relative to inner channel 201, and third gasket 128C may be configured to inhibit the low pressure fluid B from flowing (upward) beyond a location of third gasket 128B relative to inner channel 201. With outlet port 110 positioned between second gasket 128B and third gasket 128B, the pair of gaskets 128B, 128C may cooperatively redirect the low pressure fluid B into outlet port 110 for delivery toward catheter 36.

When in the second configuration of valve assembly 200, second movable shuttle 230 may be in the second position in which locking mechanism 210 is free to extend radially-outward from cavity 208 and inward into inner channel 201 without encountering an impediment by second movable shuttle 230. Stated differently, second end 234 may be positioned relatively below cavity 208 when second movable shuttle 230 is in the second position such that locking mechanism 210 may be biased toward an extended positioned. In this instance, locking mechanism 210 may be configured to abut against second end 234 and at least partially inhibit movement of second movable shuttle 230 relative to inner channel 201, such as in an upward direction beyond cavity 208. Accordingly, locking mechanism 210 may be configured to maintain second movable shuttle 230 in the second position.

Referring now to FIG. 6C, valve assembly 300 may transition to a third configuration upon releasing actuation mechanism 30 (FIG. 1). In this instance, first movable shuttle 220 and second movable shuttle 230 may move to a third position relative to valve body 202. In the third position, first movable shuttle 220 may move relative to valve body 202 while second movable shuttle 230 may remain stationary relative to valve body 202. In some examples, the third position of second movable shuttle 230 may be substantially similar to the second position of second movable shuttle 230 (FIG. 6B), and the third position of first movable shuttle 220 may be substantially similar to the first position of first movable shuttle 220 (FIG. 6A), such that releasing actuation mechanism 30 may return first movable shuttle 220 to the first position.

For example, ceasing actuation of actuation mechanism 30 may move cam 40 away from second end 224, thereby disengaging first movable shuttle 220. In this instance, biasing mechanism 226 may be returned to the first (expanded) configuration in which the upward (longitudinal) force applied against second end 224 by biasing mechanism 226 may cause first movable shuttle 220 to move (upward) relative to valve body 202 and through inner channel 201. As described in detail above, locking mechanism 210 may help to prevent second movable shuttle 230 from returning to the first position.

In the third position of the movable shuttle, first movable shuttle 220 may be repositioned at the upward-most extent relative to valve body 202 and second movable shuttle 230 may remain in the downward-most extent. When in the third configuration of valve assembly 200, first gasket 128A may remain between first (outlet) conduit 111 and second (inlet) conduit 113, second gasket 128B may be repositioned between second (inlet) conduit 113 and outlet port 110, and third gasket 128C may remain between outlet port 110 and second opening 207. Accordingly, second gasket 128B may be positioned to inhibit fluid communication between second (inlet) conduit 113 and outlet port 110.

Still referring to FIG. 6C, in the second position of second movable shuttle 230, piercing element 130 may remain at the downward-most extent relative to valve body 202 such that piercing element 130 remains in contact with seal 54 as the movable shuttle moves from the second position to the third position. Accordingly, in the third configuration of valve assembly 200, piercing element 130 may continue to extend through seal 54 such that the high pressure fluid A stored within containment device 50 may continue to be released into valve body 202 and received within inner channel 201.

The high pressure fluid A released from containment device 50 may travel along the first fluid path through inner channel 201 until encountering first gasket 128A positioned relatively above first (outlet) conduit 111. First gasket 128A may be configured to help inhibit the high pressure fluid A from flowing (upward) beyond a location of first gasket 128A relative to inner channel 201. Accordingly, first gasket 128A may redirect the high pressure fluid A into first (outlet) conduit 111 and into regulator 60. The low pressure fluid B may exit regulator 60 and travel along the second fluid path through second (inlet) conduit 113 until entering inner channel 201 when the high pressure A is reduced.

Still referring to FIG. 6C, with first movable shuttle 220 at the third position, second gasket 128B may be positioned between second (inlet) conduit 113 and outlet port 110, and third gasket 128C may remain between outlet port 110 and second opening 207. Accordingly, second gasket 128B may be positioned to inhibit fluid communication between second (inlet) conduit 113 and outlet port 110. Accordingly, the low pressure fluid B may enter inner channel 201 between first gasket 128A and second gasket 128B. First gasket 128A may be configured to inhibit the low pressure fluid B from flowing (downward) beyond a location of first gasket 128B relative to inner channel 201, and second gasket 128B may be configured to inhibit the low pressure fluid B from flowing (upward) beyond a location of second gasket 128B relative to inner channel 201. With outlet port 110 positioned above second gasket 128B, second gasket 128B may fluidly seal outlet port 110 to inhibit delivery of the low pressure fluid B toward catheter 36.

It should be appreciated that second movable shuttle 230 may remain in the second position (FIG. 6C) upon subsequent actuations of actuation mechanism 30. In other embodiments of valve assembly 200, one or more of first movable shuttle 220, second movable shuttle 230, and/or piercing element 130 may be stationary and/or fixed relative to valve body 202, such that containment device 50 may be configured to move relative to valve assembly 200 for providing a controlled release of pressurized fluid from dispensing system 10.

It should be appreciated that with the high pressure fluid A isolated from interacting with first movable shuttle 220 when valve assembly 200 is in the third configuration, due to a position of second movable shuttle 230 sealing the high pressure fluid A below the first gasket 128A, the high pressure fluid A is inhibited from affecting (e.g., increasing) the force required for a user to actuate actuation mechanism 30. Stated differently, valve assembly 200 may be operable to help prevent the high pressure fluid A from interacting with first movable shuttle 220 to promote a consistent force requirement for moving first movable shuttle 220 from the first and/or third positions (FIGS. 5A and 5C) to the second position (FIG. 5B).

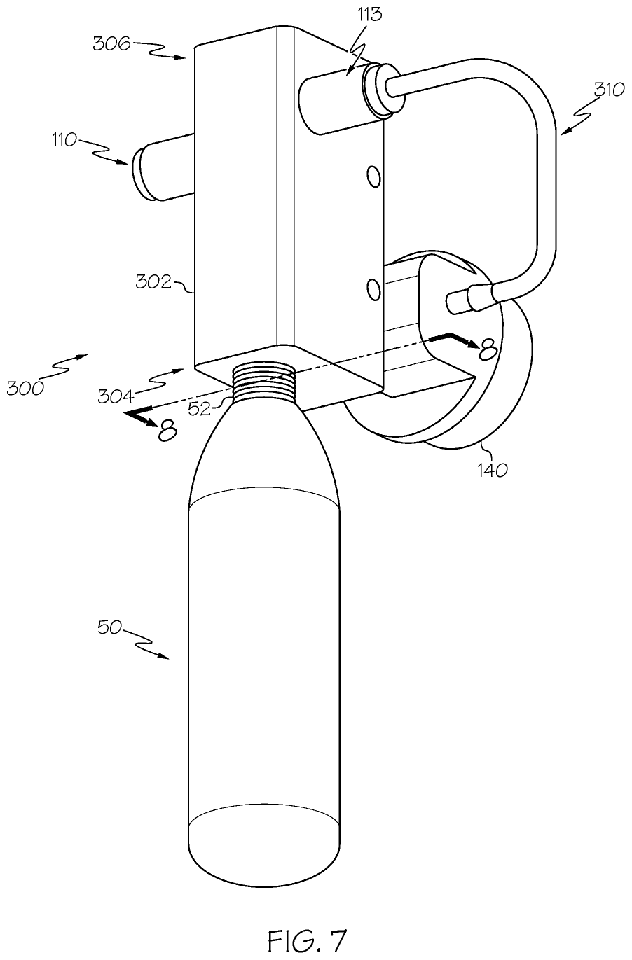

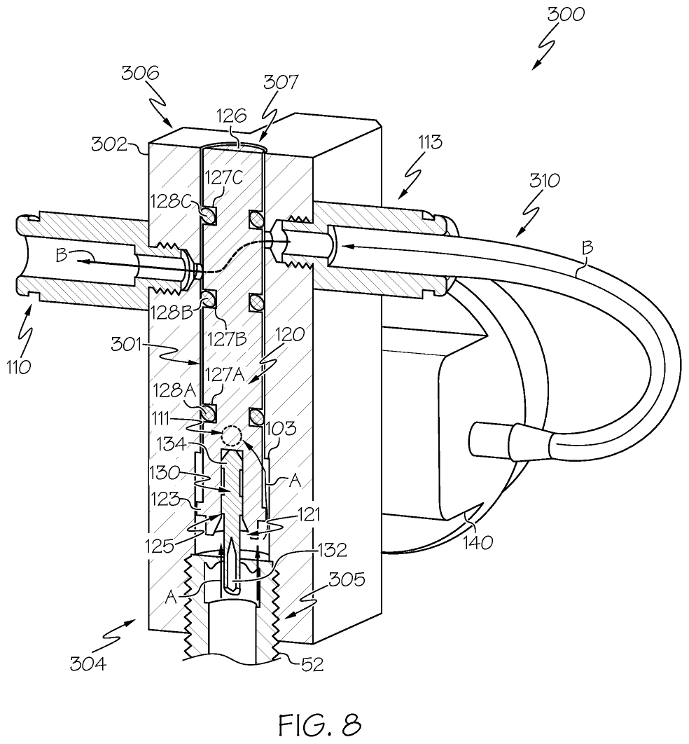

Referring now to FIGS. 7-8, aspects of another exemplary valve assembly 300 are shown. Valve assembly 300 may be configured similar to valve assembly 100, 200 except for the differences explicitly described herein, such that like reference numerals are used to identify like components. Valve assembly 300 may be housed within body 12 of delivery system 10, and selectively actuated by actuation mechanism 30 (see FIG. 1). Valve assembly 300 may include a valve body 302 having a first end portion 304 and a second end portion 306. As shown in FIG. 8, first end portion 304 may have a first opening 305, and second end portion 306 may have a second opening 307. Valve assembly 300 may be fluidly coupled to containment device 50 at first end portion 304 via first opening 305, and to regulator 60 (FIG. 1) via adapter 140.

Still referring to FIG. 8, valve assembly 300 may include an inner channel 301 extending through valve body 302 between first opening 305 in first end portion 304 and second opening 307 in second end portion 306 for receiving movable shuttle 120 and piercing element 130. Inner channel 301 may be in fluid communication with adapter 140 via first (outlet) conduit 111 and second (inlet) conduit 113. Inner channel 301 may be in further fluid communication with outlet 34 (see FIG. 1) via outlet port 110.

Valve assembly 300 may be configured and operable similar to valve assembly 100 described in detail above. For example, each of movable shuttle 120 and piercing element 130 may be configured to move between a respective plurality of positions (e.g., a first position, a second position, and a third position) to transition valve assembly 300 between a corresponding plurality of configurations (e.g., a first configuration, a second configuration, and a third configuration), similar to those shown and described in detail above with respect to valve assembly 100 (see FIGS. 5A-5C).

As seen in FIG. 8, valve assembly 300 may be in a second configuration when each of movable shuttle 120 and piercing element 130 are moved to a respective downward-most extent relative to valve body 302, thereby establishing fluid communication between inner channel 301 and containment device 50. Given a position of first gasket 128A relative to inner channel 301, the high pressure fluid A stored in containment device 50 may travel along a first fluid path through inner channel 301, first (outlet) conduit 111, and into regulator 60 via adapter 140. The low pressure fluid B released from regulator 60 may travel along a second fluid path through adapter 140, into second (inlet) conduit 113 via a tubing 310, and into inner channel 301.

In the second configuration of valve assembly 300, second gasket 128B and third gasket 128C may be cooperatively configured to direct the low pressure fluid B toward outlet port 110 for delivery to a patient due to a respective position of the gaskets 128B, 128C relative to second (inlet) conduit 113 and outlet port 110. It should be appreciated that valve assembly 300 may include a first configuration and a third configuration that are substantially similar to the first configuration (FIG. 5A) and third configuration (FIG. 5C) of valve assembly 100.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A valve assembly for a medical device, comprising:
a body having an inlet that is in fluid communication with a source of fluid, and an outlet that is in fluid communication with a delivery conduit of the medical device; and
a shuttle configured to move within the body between a first position and a second position to control delivery of the fluid from the inlet to the outlet;
wherein, in the first position, the shuttle is configured to maintain the fluid at a first pressure within the body and seal the outlet from the inlet, such that the delivery conduit is isolated from the fluid received from the source at the first pressure; and
wherein, in the second position, the shuttle is configured to maintain the fluid at a second pressure within the body and fluidly couple the outlet to the inlet, such that the delivery conduit receives the fluid from the source at the second pressure, the second pressure being less than the first pressure.

2. The valve assembly of claim 1, further comprising a piercing element configured to move within the shuttle between a retracted position and an extended position to fluidly couple the inlet with the source of fluid.

3. The valve assembly of claim 2, wherein the piercing element includes a needle or a lance having a sharp tip.

4. The valve assembly of claim 2, wherein the valve assembly is configured to receive the fluid from the source through the inlet when the shuttle is moved from the first position to the second position, and the piercing element is positioned in the retracted position; and
wherein the valve assembly is configured to inhibit delivery of the fluid from the inlet to the outlet when the shuttle is moved from the second position to the first position, and the piercing element is positioned in the extended position.

5. The valve assembly of claim 4, wherein, in a first configuration of the valve assembly:
the shuttle is positioned at the first position;
the piercing element is in the retracted position and separated from the source of fluid; and
the inlet is not in fluid communication with the outlet such that the fluid is maintained in the source of the fluid at the first pressure.

6. The valve assembly of claim 5, wherein, in a second configuration of the valve assembly:
the shuttle is positioned at the second position;
the piercing element is in the retracted position and in contact with the source of fluid; and
the inlet is in fluid communication with the outlet such that the fluid is directed to the outlet at the second pressure.

7. The valve assembly of claim 6, wherein, in a third configuration of the valve assembly:
the shuttle is positioned at the first position;
the piercing element is in the extended position and in contact with the source of fluid; and
the inlet is not in fluid communication with the outlet such that the fluid is maintained in the body at the first pressure and the second pressure.

8. The valve assembly of claim 1, wherein the body is in fluid communication with a regulator assembly configured to convert the fluid from the first pressure to the second pressure.

9. The valve assembly of claim 1, wherein the shuttle includes a gasket positioned about an exterior surface of the shuttle.

10. The valve assembly of claim 9, wherein, in a first configuration of the valve assembly:
the shuttle is positioned at the first position; and
the gasket is positioned between the inlet and the outlet to fluidly isolate the delivery conduit from the source of fluid.

11. The valve assembly of claim 10, wherein, in a second configuration of the valve assembly:
the shuttle is positioned at the second position; and
the gasket is not positioned between the inlet and the outlet to fluidly couple the delivery conduit to the source of fluid.

12. The valve assembly of claim 9, wherein the body includes a channel configured to receive the shuttle, and a ledge extending radially-inward into the channel;
wherein the shuttle includes a flange extending radially-outward from an exterior surface of the shuttle.

13. The valve assembly of claim 12, wherein the flange is configured to engage the ledge when the shuttle is in the first position relative to the channel to position the gasket between the inlet and the outlet, thereby fluidly isolating the delivery conduit from the source of fluid.

14. The valve assembly of claim 12, wherein the flange is configured to disengage the ledge when the shuttle is in the second position relative to the channel to position the gasket outside of the inlet and the outlet, thereby fluidly coupling the delivery conduit from the source of fluid.

15. The valve assembly of claim 1, further comprising a biasing mechanism coupled to the shuttle, the biasing mechanism being configured to move the shuttle from the second position to the first position.

16. A device for delivering an agent, comprising:
an enclosure configured to store an agent;
a pressurized fluid source configured to store a pressurized fluid;
a valve assembly, including:
a body having a first channel;
a shuttle configured to move within the first channel between a first position and a second position to fluidly couple the pressurized fluid source to the enclosure, the shuttle having a second channel; and
a piercing element configured to move within the second channel between a retracted position and an extended position to fluidly couple the pressurized fluid source to the body;
wherein the valve assembly is configured to selectively release the pressurized fluid from the pressurized fluid source when moving the shuttle to the second position and the piercing element to the retracted position, or moving the shuttle to the first position and the piercing element to the extended position.

17. The device of claim 16, wherein the valve assembly includes a biasing mechanism configured to bias the shuttle toward the first position when in the second position.

18. The device of claim 16, wherein the pressurized fluid from the pressurized fluid source is configured to bias the shuttle toward the first position when in the second position.

19. The device of claim 16, wherein the valve assembly is configured to selectively inhibit delivery of the pressurized fluid from the pressurized fluid source to the enclosure when moving the shuttle from the second position to the first position, and the piercing element to the extended position.

20. A method for delivering a fluid from a medical device, comprising:
moving a shuttle relative to a valve body from a first position that is offset from a source of fluid coupled to the valve body to a second position that is in contact with the source of fluid, thereby releasing the fluid into the valve body;
directing the fluid from the source to an inlet of the valve body based on the second position of the shuttle, wherein a pressure of the fluid is adjusted from a first pressure to a second pressure that is less than the first pressure when directed through the inlet;
directing the fluid from the inlet to an outlet of the valve body based on the second position of the shuttle, wherein the outlet is in fluid communication with a delivery conduit of the medical device such that the fluid at the second pressure is received at the delivery conduit;
moving the shuttle relative to the valve body from the second position to the first position, wherein the shuttle is configured to continue releasing the fluid into the valve body when in the first position; and
inhibiting the fluid from entering the outlet of the valve body based on the first position of the shuttle, such that the fluid at the first pressure and the second pressure is maintained within the valve body.
